(19) United States
(12) Patent Application Publication
(10) Pub. No.: US 2024/0386251 A1
(43) Pub. Date: Nov. 21, 2024

(54) HUMAN MOBILITY PREDICTION METHOD BASED ON GENERATIVE ADVERSARIAL NETWORK

(71) Applicant: ZHEJIANG UNIVERSITY, ZHEJIANG (CN)

(72) Inventors: Chao LI, Zhejiang (CN); Kehan LI, Zhejiang (CN); Jiming CHEN, Zhejiang (CN); Shibo HE, Zhejiang (CN); Qinmin YANG, Zhejiang (CN)

(73) Assignee: ZHEJIANG UNIVERSITY, ZHEJIANG (CN)

(21) Appl. No.: 18/267,786

(22) PCT Filed: Aug. 11, 2022
(86) PCT No.: PCT/CN2022/111718

§ 371 Date: Jun. 16, 2023

(30) Foreign Application Priority Data
Aug. 9, 2022 (CN) 202210948304.5

(51) Int. Cl. G06N 3/0475; G06N 3/045; G06N 3/094; G16H 50/80

(52) U.S. Cl. CPC G06N 3/0475; G06N 3/045; G06N 3/094; G16H 50/80

(57) ABSTRACT

The present invention discloses a human mobility prediction method based on a generative adversarial network. According to the method, the integration of spatio-temporal features of multimodal data is studied by virtue of studying urban human mobility prediction during the pandemic. A human mobility mode is difficult to be predicted during the pandemic due to complex multimodal data such as complicated social background, policy and pandemic situation. The analysis of human mobility data from three cities in China shows that different cities have highly similar human mobility modes during the pandemic, despite of great differences between them. On this basis, the disclosure designs a prediction model, in which the effects of the multimodal data on the spatio-temporal features of the human mobility are modeled integrally. In addition to this, the model can help the government evaluate the potential effects of different policies on human mobility better, and optimize policy development.

### BACKGROUND

### Technical Field

The present invention belongs to the fields of human mobility prediction, and particularly to a human mobility prediction method based on a generative adversarial network. The human mobility prediction method during the pandemic can help understand how a human mobility mode changes during the COVID-19 pandemic, especially revels that the trip restriction policies and related statistics (such as confirmed cases) issued by the government dominates human mobility rules during the COVID-19 pandemic, and precisely predicts human mobility levels.

### Description of Related Art

It is very vital for controlling the spread of the epidemic to understand how a human mobility mode changes during the COVID-19 pandemic. Taking into account the complicated social background, differences in individual behavior and extremely limited data, the human mobility mode during the pandemic seems unpredictable.

The rational policies are difficult to be developed if human mobility rules cannot be predicted precisely. Strict epidemic prevention policies will greatly affect the economy, while loose epidemic prevention policies will difficultly affect the epidemic, affecting the people's life and health. Hence, the accurate policy-making reference is grounded in the precise prediction.

Now, an urban human mobility prediction model pattern based on a deep learning model is mainly trained depending on rich historical data, and then can output a definitive prediction result by combining given current and previous observation data. However, there are limitations to this method. First, a definitive prediction model cannot generate a human mobility probability estimate; and besides, the generated result is relatively fixed, so that it is inconvenient to change some conditions flexibly for multi-dimension simulation. During the COVID-19 pandemic, the government policy-maker needs the more flexible models, including a generative model capable of learning data distribution and simulating various potential human mobility rules under different policies. The government can develop staged reopening plans based on the simulated human mobility response results during the pandemic. Hence, the applicant provides a conditional generative adversarial network for complicated dynamic modeling among new confirmed cases, policies and human mobility.

### SUMMARY

The present invention is intended to provide a human mobility prediction method based on a generative adversarial network for improving and standardizing the defects of the existing studies and technologies. The method is configured to accurately predict a human mobility variation trend by modeling the effects of different policies and pandemic situations during the pandemic, delivering a high practical value; and is conducive to analyzing potential policy effects based on data prediction results, providing a high method application scalability.

An objective of the present invention may be implemented by the following technical solution.

A human mobility prediction method based on a generative adversarial network, the method comprising the following steps:
step 1, dividing a city into H×W equal-area grids, wherein each of the grids expresses an area of the city;
step 2, dividing the areas in the step 1, and respectively counting human mobility levels m of different areas;
step 3, using the human mobility levels of the areas counted in the step 2 to obtain a human mobility map M∈R^{H×W} of the areas in the city, wherein each element in a matrix expresses the human mobility level of the corresponding area;
step 4, collecting daily statistics and relevant policies from different regions during the pandemic to obtain daily new confirmed cases C as representative statistics during the pandemic, acquiring changes and intensities of daily policies, and denoting an intensive variable of these polices as P; and
step 5, predicting a human mobility level map {Mₜ₊₁} for some time to come depending on a specific city, a given history, a current human mobility map {Mₜ₋₁, Mₜ}, and corresponding statistics {Cₜ₋₁, Cₜ, Cₜ₊₁} and policies {Pₜ₋₁, Pₜ} on the COVID-19 pandemic.

Further, in the step 5, the human mobility level map for some time to come is generated by predicting a human mobility rule during the pandemic through a human mobility prediction model during the pandemic; the human mobility prediction model during the pandemic comprises a generator module, a discriminator module and a domain knowledge fusion module,
wherein the generator module is used for predicting the human mobility rule for some time to come based on the historical human mobility data for some time past;
the discriminator module is used for predicting a label of the human mobility map and determining whether the generated human mobility map is consistent with the real human mobility distribution; and
the domain knowledge fusion module is used for integrating the effects of external factors during the pandemic.

Further, the generator module models the responses of human mobility intensities on policy changes during the pandemic in different areas by modeling human mobility change values between varying time steps, and an input of the generator module is denoted as a human mobility level between two time segments:

$$Δ {M}_{t - 1} = {M}_{t} - {M}_{t - 1} .$$

Further, a transformer encoder module is introduced into the human mobility prediction model during the pandemic to model a long-distance spatial-temporal correlativity, a multi-head self-attention mechanism module is introduced to extract a feature map f₀∈ [symbol] ^{H×W×C}, a transformer-processed feature is denoted as f₁∈ [symbol] ^{H×W×C}, and output features and external conditions are spliced and delivered to a human mobility result output and predicted by a decoder.

Further, the domain knowledge fusion module integrates the policies and the pandemic statistics during the pandemic as conditions with spatio-temporal human mobility features, specifically including: introducing a fully-connected neural network, converting different kinds of domain knowledge into a hidden variable C∈R^{H×W×c}^{₀}, and then introducing a gated fusion network module to activate the spatio-temporal features of different areas:

$${f}_{1}^{′} = σ ( C ⊗ {f}_{1} )$$

the human mobility prediction model during the pandemic introduces a noise vector N∈R^{H×W×n}^{₀} and a spatio-temporal feature vector for splicing in a feature dimension in a working space, and finally introduces a cross-model connector into the human mobility prediction model during the pandemic to obtain a human mobility level estimate of next time step:

$${M}_{t + 1} = {M}_{t} + Δ {\hat{M}}_{t} .$$

Further, the human mobility prediction model during the pandemic introduces a mask matrix K∈R^{H×W} to reduce the effects from the areas lack of sampling, thus enabling to calculate a loss function with the mask matrix from the generator module:

$${L}_{G} = 1 / {‖ K ‖}_{1 , 1} {‖ ( {\hat{M}}_{t + 1} - {M}_{t + 1} ) ⊙ K ‖}_{F}^{2} ,$$

whereby a loss function of the discriminator module is obtained as follows:

$${L}_{D} = {E}_{M ∼ {p}_{d a t a}} log D ( {M}_{t + 1} , {M}_{t} ) + {E}_{N ∼ {p}_{N}} log ( 1 - D ( G ( {M}_{t} , {M}_{t + 1} , C , N ) ) ) ,$$

and finally combining the loss functions of the generator module and the discriminator module to obtain a formula as follows:

$${min}_{G} {max}_{D} {L}_{G} + λ {L}_{D} ,$$

and training the modules to reach the saddle points of the loss functions of the generator module and the discriminator module, which indicates that model training is completed.

Further, the daily policy comprises one or more indicators of the policies such as trip restriction and lockdown, economy and health system.

Compared with the prior art. the present invention has the following beneficial effects:
1) aprogressive learning style according to the present invention can be applied to dealing with the fast changing human mobility data during the pandemic;
2) the domain knowledge fusion module according to the present invention can be introduced to model the effects of the policies and pandemic development situation on the human mobility change; and
3) the generator module and the discriminator module according to the present invention constitute the generative adversarial network for modeling the uncertainty during the human mobility.

### BRIEF DESCRIPTION OF THE DRAWINGS

FIG. 1 is a flow chart according to the present invention; and

FIG. 2 is a schematic structural diagram of a human mobility prediction model during the pandemic according to the present invention.

### DESCRIPTION OF THE EMBODIMENTS

It should be understood that, in the description of the present invention, the orientation or position relations indicated by the terms “one end”, “another end”, “outside”, “on”, “inside”, “horizontal”, “coaxial”, “center”, “end”, “length”, and “outer end”, etc. are presented merely for describing the present invention and simplifying the description, instead of indicating or implying that the referred device or element must have special orientation, or configured and operated at a special orientation. Therefore, all of them cannot be understood as the limits to the present invention.

The present invention is further explained with reference to the accompanying drawings.

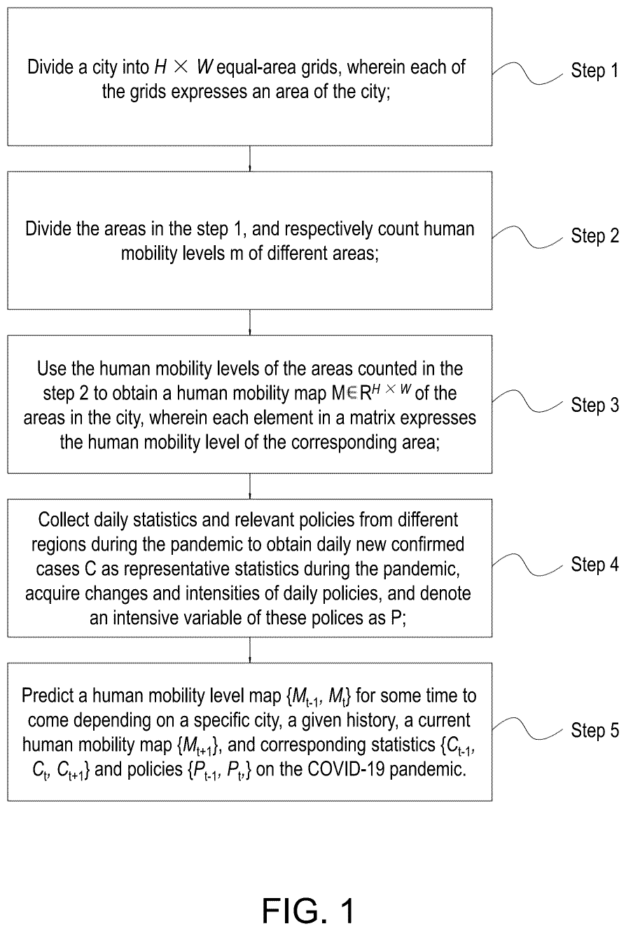

By referring to FIG. 1. a human mobility prediction method based on a generative adversarial network, the method comprising the following steps:
step 1, dividing a city into H×W equal-area grids, wherein each of the grids expresses an area of the city;
step 2, dividing the areas in the step 1, and respectively counting human mobility levels m of different areas;

step 3, using the human mobility levels of the areas counted in the step 2 to obtain a human mobility map M∈R^{H×W} of the areas in the city, wherein each element in a matrix expresses the human mobility level of the corresponding area;

step 4, collecting daily statistics and relevant policies from different regions during the pandemic to obtain daily new confirmed cases C as representative statistics during the pandemic, acquiring changes and intensities of daily policies, and denoting an intensive variable of these polices as P;

step 5, predicting a human mobility level map {Mₜ₊₁} for some time to come depending on a specific city, a given history, a current human mobility map {Mₜ₋₁, Mₜ}, and corresponding statistics {Cₜ₋₁, Cₜ, Cₜ₊₁} (many articles have proven accurate real-time prediction on new confirmed cases) and policies {Pₜ₋₁, Pₜ} (developed by decision-makers in advance) on the COVID-19 pandemic.

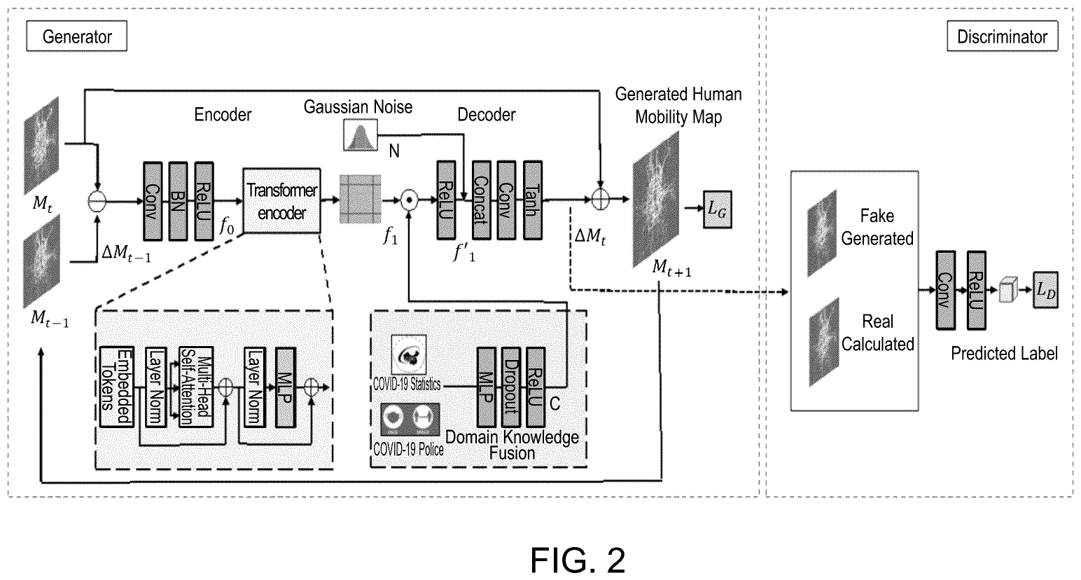

With reference to FIG. 2, in the step 5, the human mobility level map for some time to come is generated by predicting a human mobility rule during the pandemic through a human mobility prediction model during the pandemic; the human mobility prediction model during the pandemic comprises a generator module, a discriminator module and a domain knowledge fusion module, wherein the generator module is used for predicting the human mobility rule for some time to come based on the historical human mobility data for some time past; the discriminator module is used for predicting a label of the human mobility map and determining whether the generated human mobility map is consistent with the real human mobility distribution; and the domain knowledge fusion module is used for integrating the effects of external factors during the pandemic.

The generator module and the discriminator module constitute the generative adversarial network for modeling the uncertainty during the human mobility.

The human mobility is estimated by referring to the policies and statistics on new confirmed cases during the pandemic. The present invention provides a rule for using a model based on a deep generative network to predict human mobility during the pandemic. A generative model capable of learning data distribution and simulating various potential human mobility rules under different policies. The government can develop staged reopening plans based on the simulated human mobility response results during the pandemic. The present invention provides a conditional generative adversarial network for complicated dynamic modeling among new confirmed cases, policies and human mobility. Different from modeling a long-term time sequence dependent relation by a traditional model, the model focuses on predicting human mobility changes between the adjacent time segments formed by potential pandemic statistics, policies and latest human mobility trends. To be specific, the present invention designs a policy-human mobility interplay network (PHMIN) model to estimate the human mobility changes. Here, model condition inputs are mainly from the statistics and policies during the pandemic. The model can flexibly learn fine-grained human mobility trends, and accurately expand to the prediction on many waves of cross-city pandemics. See FIG. 1 for the model structure.

Further, the generator module efficiently models the responses of human mobility intensities on policy changes during the pandemic in different areas by modeling human mobility change values between varying time steps, and an input of the generator module is denoted as a human mobility level between two time segments: ΔMₜ₋₁=Mₜ−Mₜ₋₁.

Further, considering another aspect of the multi-scale spatio-temporal human mobility features, a transformer encoder module is introduced here to model a long-distance spatial-temporal correlativity, a multi-head self-attention mechanism module is introduced to extract a feature map f₀∈R^{H×W×C}, a transformer-processed feature is denoted as f₁∈R^{H×W×C}, and output features and external conditions are spliced and delivered to a human mobility result output and predicted by a decoder.

Further, the domain knowledge fusion module integrates the policies and the pandemic statistics during the pandemic as conditions with spatio-temporal human mobility features, specifically including: introducing a fully-connected neural network, converting different kinds of domain knowledge into a hidden variable C∈R^{H×W×c}^{₀}, and then introducing a gated fusion network module to activate the spatio-temporal features of different areas:

$${f}_{1}^{′} = σ ( C ⊗ {f}_{1} ) ,$$

and meanwhile considering the uncertainty during the human mobility prediction, the human mobility prediction model during the pandemic introduces a noise vector N∈ [symbol] ^{H×W×n}^{₀} and a spatio-temporal feature vector for splicing in a feature dimension in a working space, and finally introduces a cross-model connector into the human mobility prediction model during the pandemic to obtain a human mobility level estimate of next time step:

$${M}_{t + 1} = {M}_{t} + Δ {\hat{M}}_{t} .$$

Further, the human mobility prediction model during the pandemic introduces a mask matrix K∈ [symbol] ^{H×W} during the model optimization to reduce the effects from the areas lack of sampling, thus enabling to calculate a loss function with the mask matrix from the generator module:

$${L}_{G} = 1 / {‖ K ‖}_{1 , 1} {‖ ( {\hat{M}}_{t + 1} - {M}_{t + 1} ) ⊙ K ‖}_{F}^{2} ,$$

further whereby a loss function of the discriminator module is obtained as follows:

$${L}_{D} = {E}_{M ∼ {p}_{d a t a}} log D ( {M}_{t + 1} , {M}_{t} ) + {E}_{N ∼ {p}_{N}} log ( 1 - D ( G ( {M}_{t} , {M}_{t + 1} , C , N ) ) ) ,$$

and finally combining the loss functions of the generator module and the discriminator module to obtain a formula as follows:

$${min}_{G} {max}_{D} {L}_{G} + λ {L}_{D} ,$$

and training the modules to reach the saddle points of the loss functions of the generator module and the discriminator module, which indicates that model training is completed.

Further, the daily policy comprises one or more indicators of the policies such as trip restriction and lockdown, economy and health system.

### Embodiments

In an embodiment, the human mobility prediction method during the pandemic is applied to predicting the human mobility during the pandemic in Beijing, Dalian and Shijiazhuang, among which BJ expresses Beijing, DL expresses Dalian, and SJZ expresses Shijiangzhuang. Numbers behind indicate human mobility data during the pandemics of different waves. Considering seven waves of COVID-19 pandemics in the three cities, the pandemic is divided into two intensities for three groups of experiments according to actual conditions in the embodiment: 1) experiments under similar intensities in varying cities; 2) experiments under different intensities in varying cities; and 3) layered analysis of experiment results in the scenarios with similar or different intensities in the same city. These experiments are designed to answer a basic question: can the model be effectively generalized in varying environments for predicting the effects on human mobility when a next wave of pandemic comes. The following paragraphs will discuss this question in detail.

TABLE 6.1
Cross-city Prediction Experiments under Similar Pandemic Intensities

| Model | HA | | ARIMA | | cGAN | | DccpST | | PHMIN | |
|---|---|---|---|---|---|---|---|---|---|---|
| Setting | MAE | MAPE | MAE | MAPE | MAE | MAPE | MAE | MAPE | MAE | MAPE |
| BJ1-DL1 | 1260 | 0.111 | 1013 | 0.086 | 2639 | 0.181 | 3773 | 0.255 | 629 | 0.058 |
| SJZ1-DL1 | 1260 | 0.111 | 1013 | 0.086 | 1782 | 0.170 | 2617 | 0.187 | 648 | 0.062 |
| DL1-BJ1 | 10305 | 0.369 | 6558 | 0.177 | 38316 | 1.337 | 32221 | 1.015 | 5803 | 0.222 |
| SJZ1-BJ1 | 10305 | 0.369 | 6558 | 0.177 | 22947 | 0.752 | 15752 | 0.501 | 5412 | 0.177 |
| BJ1-SJZ1 | 1824 | 0.201 | 1138 | 0.115 | 2413 | 0.238 | 3704 | 0.309 | 842 | 0.091 |
| DL1-SJZ1 | 1824 | 0.201 | 1138 | 0.115 | 3368 | 0.497 | 3745 | 0.442 | 876 | 0.128 |
| DL1-SJZ2 | 2221 | 0.368 | 1599 | 0.263 | 3760 | 0.805 | 3622 | 0.578 | 1349 | 0.273 |
| BJ1-SJZ2 | 2221 | 0.368 | 1599 | 0.263 | 3012 | 0.371 | 3368 | 0.343 | 1876 | 0.253 |
| BJ2-DL2 | 1136 | 0.063 | 1090 | 0.064 | 1397 | 0.111 | 1623 | 0.146 | 554 | 0.038 |
| BJ2-DI3 | 1366 | 0.093 | 1012 | 0.077 | 1439 | 0.125 | 1924 | 0.158 | 947 | 0.062 |

Similar intensities in varying cities: According to the experiment results as shown in Table 6.1, the following conclusion can be obtained.

In conclusion, the PHMIN model can achieve the best performance compared with a baseline model. This certifies that the model has a good generalization ability in the cross-city human mobility prediction scenarios. To be specific, rather simple HA and ARIMA models show better DeepST and cGAN performance, which means the most valuable information and latest human mobility conditions in the question to a certain degree. Besides, the cGAN is slightly superior to the DeepST on account that cGAN can model unknown human mobility factors generatively. On the other hand, the definitive prediction based on rich historical observations given by the DeepST is not appropriate due to the lack of historical data in the pandemic scenario.

Based on a same given test city as a prediction target, it is found that the models trained by varying cities will have different manifestations. When testing on DL1, SJZ1 and SJZ2, a model trained based on BJ1 shows the better effect than other cities. This can contribute to higher data quality, more available samples and higher crowd density in Beijing.

The urban geographic distribution similarity may affect the cross-city prediction effect of the models. It is found, through the experiment results, that the model trained based on DL1generally results the poorer performance. Taking the results of DL1-BJ1, DL1-SJZ1 and DL1-SJZ2 for example, their results have no significant difference from the traditional ARIMA (MAE is better, MAPE is poorer). In fact, Dalian urban area is surrounded by the sea, with irregular geological distribution, while Beijing and Shijiazhuang urban areas have a very regular and chessboard-like layout, respectively. It can be inferred from this that the differences in spatial distribution will possibly result in more difficult model generalization between the cities.

TABLE 6.2
Cross-city Prediction Experiments under Different Cross-city Intensities

| Model | HA | | ARMIA | | cGAN | | DeepST | | PHMIN | |
|---|---|---|---|---|---|---|---|---|---|---|
| Setting | MAE | MAPE | MAE | MAPE | MAE | MAPE | MAE | MAPE | MAE | MAPE |
| BJ1-DL2 | 1136 | 0.064 | 1090 | 0.064 | 3923 | 0.235 | 5131 | 0.301 | 777 | 0.048 |
| SJZ1-DL2 | 1136 | 0.064 | 1090 | 0.064 | 2374 | 0.157 | 3707 | 0.225 | 805 | 0.051 |
| BJ1-DL3 | 1366 | 0.093 | 1012 | 0.077 | 3478 | 0.205 | 5619 | 0.309 | 981 | 0.066 |
| SJZ1-DL3 | 1366 | 0.093 | 1012 | 0.077 | 1958 | 0.151 | 5724 | 0.305 | 1052 | 0.070 |
| DL1-BJ2 | 7373 | 0.126 | 9411 | 0.125 | 20808 | 0.437 | 21841 | 0392 | 4503 | 0.086 |
| SJZ1-BJ2 | 7373 | 0.126 | 9411 | 0.125 | 15599 | 0.283 | 16008 | 0.287 | 4678 | 0.087 |
| BJ2-SJZ2 | 2221 | 0.369 | 1599 | 0.263 | 3423 | 0.631 | 2628 | 0.408 | 1566 | 0.242 |
| DL2-SJZ2 | 2221 | 0.369 | 1599 | 0.263 | 5332 | 1.057 | 3926 | 0.596 | 2046 | 0.348 |

Different intensities in varying cities: The experiments as shown in Table 6.2 are further designed and performed to study the generalization performance of the models between the pandemics with different intensities. The experiment result are as follows.

In most cases, the PHMIN models still show a better performance than the baseline model. However, the overall performance predicted by the training sets with different intensities is poorer than that of the models with similar intensities in Table 6.1 in the same testing city. This shows that the pandemic intensity is a more critical indicator that affects the generalization ability of the model compared with different city layouts.

The models trained based on Dalian data still show a poor performance compared with other cities (such as DL1-BJ2 and DL2-SJZ2), which illustrates the significance on similar urban geographical distribution. Besides, the proposed model has the poorer prediction result than the baseline method ARIMA in a DL2-SJZ2 scenario due to the differences in geological distribution and pandemic intensity.

TABLE 6.3
Prediction Experiments on Same City with Similar or Different Intensities

| Model | HA | | ARIMA | | cGAN | | DccpST | | PHMIN | |
|---|---|---|---|---|---|---|---|---|---|---|
| Setting | MAE | MAPE | MAE | MAPE | MAE | MAPE | MAE | MAPE | MAE | MAPE |
| SJ21QZ2 | 2221 | 0.369 | 1599 | 0.263 | 2442 | 0.395 | 2110 | 0.304 | 1457 | 0.232 |
| DL2-DL3 | 1366 | 0.093 | 1012 | 0.077 | 2066 | 0.189 | 3533 | 0.201 | 695 | 0.051 |
| BJ1-BJ2 | 7373 | 0.126 | 9411 | 0.125 | 20047 | 0.324 | 30132 | 0.496 | 3799 | 0.065 |
| DL1-DL2 | 1136 | 0.064 | 1090 | 0.064 | 2140 | 0.135 | 2706 | 0.157 | 714 | 0.042 |
| DL1-DL3 | 1366 | 0.093 | 1012 | 0.077 | 1676 | 0.133 | 1752 | 0.140 | 1050 | 0.067 |

Same city with similar or different intensities: Intuitively, the model should be easily generalized better in case of conducting the experiments in the same city. Nevertheless, the experiment data in Table 6.3 reveals some different results, as follows.

First, the PHMIN model is superior to the baseline model in all experiments. Besides, the three groups of experiments, including SJZ1-SJZ2, DL2-DL3 and BJ1-BJ2, have the better effect than the results in Tables 6.1 and 6.2. This means the negative effects from the differences in the same city may be lower than the pandemic intensity.

But the results of the DL1-DL2 and DL1-DL3 experiments are respectively poorer than of the BJ2-DL2 and BJ2-DL3 experiments. This can be ascribed to BJ2, DL2 and DL3. Three data sets have similar pandemic intensities in varying cities. It is inferred from the comparison of the results of the DL2-DL3 and DL1-DL3 experiments that the proposed model can show the better generalization performance in case of predicting the human mobility in the scenarios with the similar pandemic intensities in the same city.

Eventually, it should be noted that the above-mentioned embodiments are presented merely for describing the technical solutions of the present invention, and in no way should be considered to be limitations of the present invention. Although the present invention is depicted in detail by referring to the foregoing embodiments, those of ordinary skill in the art should understand that they can still amend the technical solutions recited in the foregoing embodiments, or equivalently replace some or all technical features therein. Nevertheless, these amendments or replacements should not make the nature of the corresponding technical solutions depart from the scope of the technical solutions of all embodiments in the present invention.

1. A human mobility prediction method based on a generative adversarial network, the method comprising following steps:
step 1, dividing a city into H×W equal-area grids, wherein each of the grids expresses an area of the city;
step 2, dividing the areas in the step 1, and respectively counting human mobility levels m of the different areas;
step 3, using the human mobility levels of the areas counted in the step 2 to obtain a human mobility map M∈R^{H×W} of the areas in the city, wherein each element in a matrix expresses the human mobility level of the corresponding area;
step 4, collecting daily statistics and relevant policies from different regions during a pandemic to obtain daily new confirmed cases C as representative statistics during the pandemic, acquiring changes and intensities of daily policies, and denoting an intensive variable of these polices as P; and
step 5, predicting a human mobility level map {Mₜ₊₁} for a period of time to come depending on a specific city, a given history, a current human mobility map {Mₜ₋₁, Mₜ}, and corresponding statistics {Cₜ₋₁, Cₜ, Cₜ₊₁} and policies {Pₜ₋₁, Pₜ} on COVID-19 pandemic.

2. The human mobility prediction method based on the generative adversarial network according to claim 1, wherein in the step 5, the human mobility level map for the period of time to come is generated by predicting a human mobility rule during the pandemic through a human mobility prediction model during the pandemic; the human mobility prediction model during the pandemic comprises a generator module, a discriminator module and a domain knowledge fusion module,
wherein the generator module is used for predicting the human mobility rule for the period of time to come based on historical human mobility data for a period of time past;
the discriminator module is used for predicting a label of the human mobility map and determining whether the generated human mobility map is consistent with real human mobility distribution; and
the domain knowledge fusion module is used for integrating effects of external factors during the pandemic.

3. The human mobility prediction method based on the generative adversarial network according to claim 2, wherein the generator module models responses of human mobility intensities on policy changes during the pandemic in different areas by modeling human mobility change values between varying time steps, and an input of the generator module is denoted as a human mobility level between two time segments:
$$Δ {M}_{t - 1} = {M}_{t} - {M}_{t - 1} .$$

4. The human mobility prediction method based on the generative adversarial network according to claim 2, wherein a transformer encoder module is introduced into the human mobility prediction model during the pandemic to model a long-distance spatial-temporal correlativity, a multi-head self-attention mechanism module is introduced to extract a feature map f₀∈ [symbol] ^{H×W×C} , a transformer-processed feature is denoted as f₁∈ [symbol] ^{H×W×C}, and output features and external conditions are spliced and delivered to a human mobility result output and predicted by a decoder.

5. The human mobility prediction method based on the generative adversarial network according to claim 2, wherein the domain knowledge fusion module integrates the policies and the statistics during the pandemic as conditions with spatio-temporal features, specifically including: introducing a fully-connected neural network, converting different kinds of domain knowledge into a hidden variable C∈ [symbol] ^{H×W×c}^{₀}, and then introducing a gated fusion network module to activate the spatio-temporal features of different areas:
$${f}_{1}^{′} = σ ( C ⊗ {f}_{1} ) ,$$
the human mobility prediction model during the pandemic introduces a noise vector N∈ [symbol] ^{H×W×n}^{₀} and a spatio-temporal feature vector for splicing in a feature dimension in a working space, and finally introduces a cross-model connector into the human mobility prediction model during the pandemic to obtain a human mobility level estimate of next time step:
$${M}_{t + 1} = {M}_{t} + Δ {\hat{M}}_{t} .$$

6. The human mobility prediction method based on the generative adversarial network according to claim 2, wherein the human mobility prediction model during the pandemic introduces a mask matrix K∈ [symbol] ^{H×W} to reduce effects from the areas lack of sampling, thus enabling to calculate a loss function with the mask matrix from the generator module:
$${L}_{G} = 1 / {‖ K ‖}_{1 , 1} {‖ ( {\hat{M}}_{t + 1} - {M}_{t + 1} ) ⊙ K ‖}_{F}^{2} ,$$
whereby a loss function of the discriminator module is obtained as follows:
$${L}_{D} = {E}_{M ∼ {p}_{data}} log D ( {M}_{t + 1} , {M}_{t} ) + {E}_{N ∼ {p}_{N}} log ( 1 - D ( G ( {M}_{t} , {M}_{t + 1} , C , N ) ) ) ,$$
and finally combining the loss functions of the generator module and the discriminator module to obtain a formula as follows:
$${min}_{G} {max}_{D} {L}_{G} + λ {L}_{D} ,$$
and training the generator module and the discriminator module to reach saddle points of the loss functions of the generator module and the discriminator module, which indicates that model training is completed.

7. The human mobility prediction method based on the generative adversarial network according to claim 1, wherein the daily policies comprise one or more indicators of the policies such as trip restriction and lockdown, economy and health system.
